(19) United States
(12) Patent Application Publication
(10) Pub. No.: US 2009/0187075 A1
(43) Pub. Date: Jul. 23, 2009

(54) GUIDE WIRE ENGAGING URETEROSCOPE

(75) Inventors: Manoj Monga, Minneapolis, MN (US); Arthur G. Erdman, New Brighton, MN (US); Ryan Buesseler, Fergus Falls, MN (US)

Correspondence Address: SCHWEGMAN, LUNDBERG & WOESSNER, P.A., P.O. BOX 2938, MINNEAPOLIS, MN, 55402, US

(73) Assignee: Regents of the University of Minnesota, St. Paul, MN (US)

(21) Appl. No.: 12/270,579

(22) Filed: Nov. 13, 2008

Related U.S. Application Data
(63) Continuation of application No. 11/417,498, filed on May 4, 2006, now abandoned.
(60) Provisional application No. 60/678,148, filed on May 5, 2005.

(51) Int. Cl. A61B 1/307

(52) U.S. Cl. 600/135

(57) ABSTRACT

This document discusses, among other things, a ureteroscope having a notch or other structure at a distal end configured to engage a guide wire and facilitate cannulation.

### CROSS-REFERENCE TO RELATED PATENT DOCUMENTS

This patent application is a continuation of U.S. patent application Ser. No. 11/417,498, filed May 4, 2006, which claims the benefit of priority, under 35 U.S.C. Section 119(e), to Monga et al., U.S. Provisional Patent Application Ser. No. 60/678,148, entitled “NOTCHED URETEROSCOPE,” filed on May 5, 2005 (Attorney Docket No. 600.648PRV), which applications are incorporated herein by reference.

### TECHNICAL FIELD

This document pertains generally to medical devices, and more particularly, but not by way of limitation, to ureteroscopes.

### BACKGROUND

The ureter is a channel that drains urine from the kidney to the bladder. A ureteroscope is a shafted instrument typically used for examining the ureter and treating ureteral stones, tumors and strictures. The ureteroscope is passed through the urethra and into the bladder. Difficulties are sometimes encountered in cannulating the ureteral orifice. The ureteral orifice is the opening in the bladder where the ureter empties urine. The orifice is often tight and intermittently contracts, thus impeding advancement of the ureteroscope. Difficulties may also be encountered in maneuvering around sites of narrowing or obstruction once the ureteroscope is in the ureter.

### BRIEF DESCRIPTION OF THE DRAWINGS

In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

FIG. 1 illustrates anatomy of the ureter.

FIGS. 2A, 2B and 2C illustrate distal ends of various ureteroscopes.

FIG. 3 illustrates a perspective view of a notched ureteroscope.

FIG. 4 illustrates an end view of a notched ureteroscope.

FIG. 5 illustrates top and end views of a notched ureteroscope.

FIG. 6 illustrates an end view of a notched ureteroscope.

FIG. 7 illustrates a magnetic ureteroscope.

FIG. 8 illustrates an end view of a dimpled ureteroscope.

FIG. 9 illustrates a perspective view of a dimpled ureteroscope.

FIG. 10 illustrates an end view of a dimpled ureteroscope in a ureter.

FIG. 11 illustrates a perspective view of a notched ureteroscope.

FIG. 12 illustrates a perspective view of a ureteroscope having an extended notch.

FIG. 13 illustrates a perspective view of a ureteroscope having a notched beak portion.

### DETAILED DESCRIPTION

The following detailed description includes references to the accompanying drawings, which form a part of the detailed description. The drawings show, by way of illustration, specific embodiments in which the invention may be practiced. These embodiments, which are also referred to herein as “examples,” are described in enough detail to enable those skilled in the art to practice the invention. The embodiments may be combined, other embodiments may be utilized, or structural, logical and electrical changes may be made without departing from the scope of the present invention. The following detailed description is, therefore, not to be taken in a limiting sense, and the scope of the present invention is defined by the appended claims and their equivalents.

A notch or other structure in the end of the shaft of the ureteroscope engages a guide wire positioned in the ureter. The notch is engaged with the guide wire and stabilizes the wire while an upward force is exerted. In addition, the notch or other structure can be used to apply a lateral or downward force on the guide wire.

The ureteroscope can be described as having a shaft that is rigid or semi-rigid. In a rigid shaft, a rod lens is disposed inside the shaft and in a semi-rigid shaft, a fiber optic element allows small deflections along the shaft length.

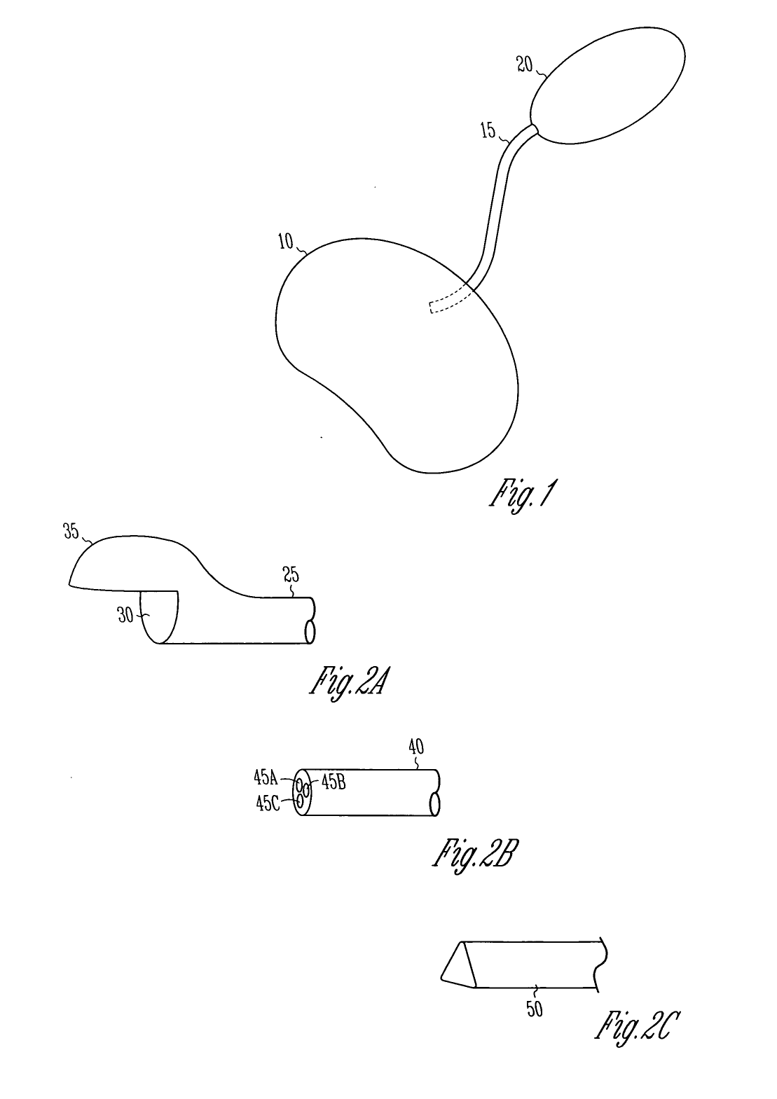

FIG. 1 illustrates bladder 10 coupled to kidney 20 via ureter 15. As part of a ureteroscopy procedure, a guide wire is temporarily placed in ureter 15 with one end coiling in the renal pelvis area and the other end extending from the ureteral orifice, through bladder 10 and out the urethra (not shown).

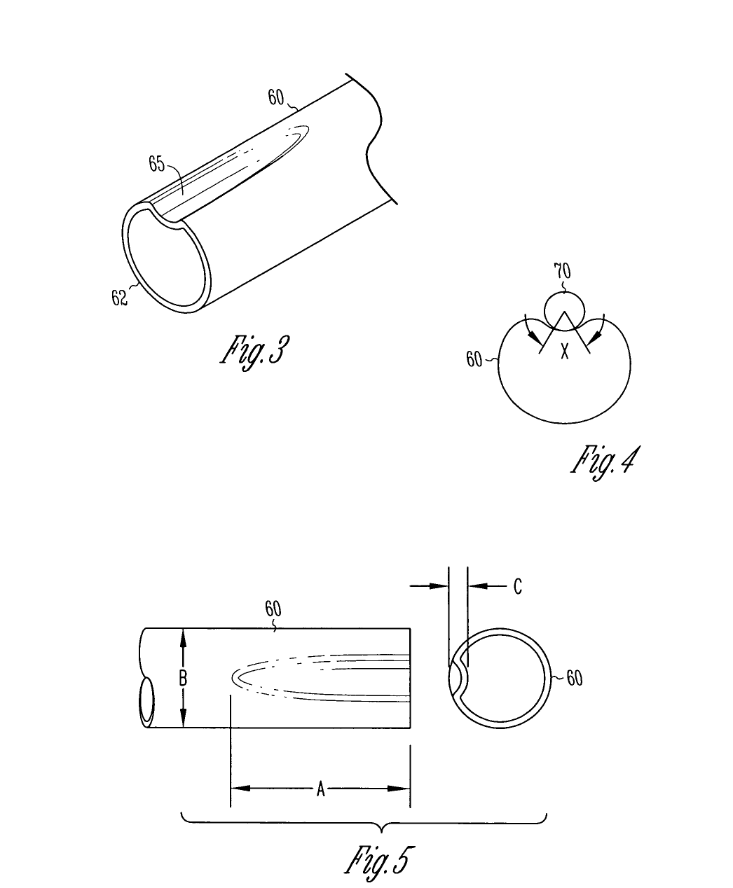

FIG. 2A illustrates a distal end of ureteroscope having shaft 25. Beak 35 is disposed on an end of shaft 25. In addition, end 30 includes one or more channels or lumens for carrying instruments or other materials through the ureteroscope.

FIG. 2B illustrates a plain or blunt end shaft 40. In the figure, channels 45A, 45B and 45C are illustrated. In various configurations, channels 45A, 45B and 45C carry such elements as a light bundle, a fiber optic imaging channel, and a working channel.

FIG. 2C illustrates shaft 50 having a triangular cross-sectional profile. Multiple channels within shaft 50 can be used and are not illustrated in this view.

FIG. 3 illustrates a perspective view of shaft 60 having notch 65. In various examples, notch 65 is formed by cutting, machining, shaping, deforming, extruding or casting shaft 60. In the figure, shaft 60 is illustrated as a hollow structure having a uniform wall thickness, however, in other examples, shaft 60 is a solid or includes one or more lumens channels and the wall thickness is non-uniform. Edge 62 has a radius that reduces injury during a ureteroscopy procedure. In addition, the edges of notch 65 are smooth and rounded to preclude injury and facilitate easy passage.

FIG. 4 illustrates an end view of ureteroscope shaft 60. In the figure, guide wire 70 is disposed in a notch on the circumference of shaft 60. The region of contact between guide wire 70 and shaft 60 is denoted by angle x and in one example, angle x is approximately 120 degrees, however, greater or lesser angles are also contemplated.

Guide wire 70, not shown to scale in the figures, typically includes a stainless steel or a shape memory alloy such as nitinol (alloy of nickel and titanium) with a coating of polytetrafluoroethylene (PTFE, commercially available under the name Teflon) or silicone. Standard sizes for guide wires are 0.035 and 0.038 inch diameter. Other guide wires are also contemplated, including, for example, a ferrous or magnetic guide wire.

FIG. 5 illustrates top and end views of notched ureteroscope shaft 60. In one example, notch length A has dimension of approximately 0.050 to 0.500 inch and notch depth C has dimension of approximately 0.020 inch on shaft diameter B of approximately 2 mm to 3 mm. Other dimensions are also contemplated. In one example, notch depth C is selected such that the region of contact is tailored to facilitate reliable engagement of the guide wire and also easy disengagement.

FIG. 6 illustrates end view of shaft 80 having elements 45A, 45B and 45C and notch 85. In the figure, notch 85 has a “v” profile and is formed by a cutting tool which forms side walls at angle λ. Other angles are also contemplated, but in one example, angle λ is approximately 85-90 degrees.

Alignment of notch 85 relative to elements 45A, 45B and 45C can be selected to suit a particular procedure or other objective. For example, in one instance an imaging element is located directly below a notch. As another example, one instance provides that an illumination element is located nearest the notch in the shaft. Other configurations are also contemplated.

FIG. 7 illustrates shaft 90 having magnet 95. Magnet 95, in various examples, includes a permanent magnet or an electromagnet. In one example, permanent magnet 95 is embedded in a distal end and is configured to lie flush with the surface of shaft 90 or recessed. In addition, a sheath can be applied over shaft 90. In operation, shaft 90 is rotated and positioned to engage a magnetically susceptible guide wire during cannulization. In one example, shaft 90 is rotated to disengage from the guide wire. In one example, magnet 95 includes an electromagnet and a user operable switch is provided to modulate the magnetic field strength to facilitate engagement and disengagement of the guide wire. Electrical conductors for operating a coil of electromagnet are routed internally or externally relative to shaft 90.

In one example, the guide wire is engaged and disengaged relative to the notch by rotating the shaft. In one example, the guide wire is engaged by retracting a sheath or a shim from between the guide wire and the magnet and disengaged by interjecting a sheath or shim between the guide wire and the magnet. In various examples, a sheath or catheter is disposed over either the guide wire, the shaft or both the guide wire and the shaft.

In one example, engagement and disengagement is controlled by changing the position or orientation of an internal magnet within the ureteroscope shaft.

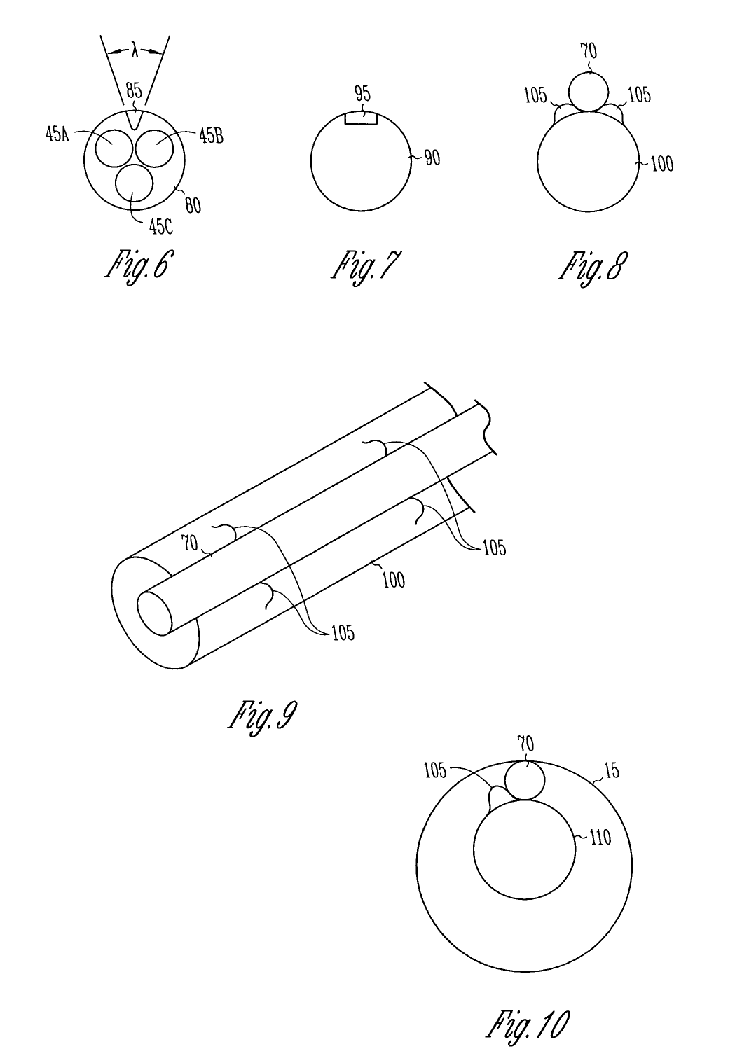

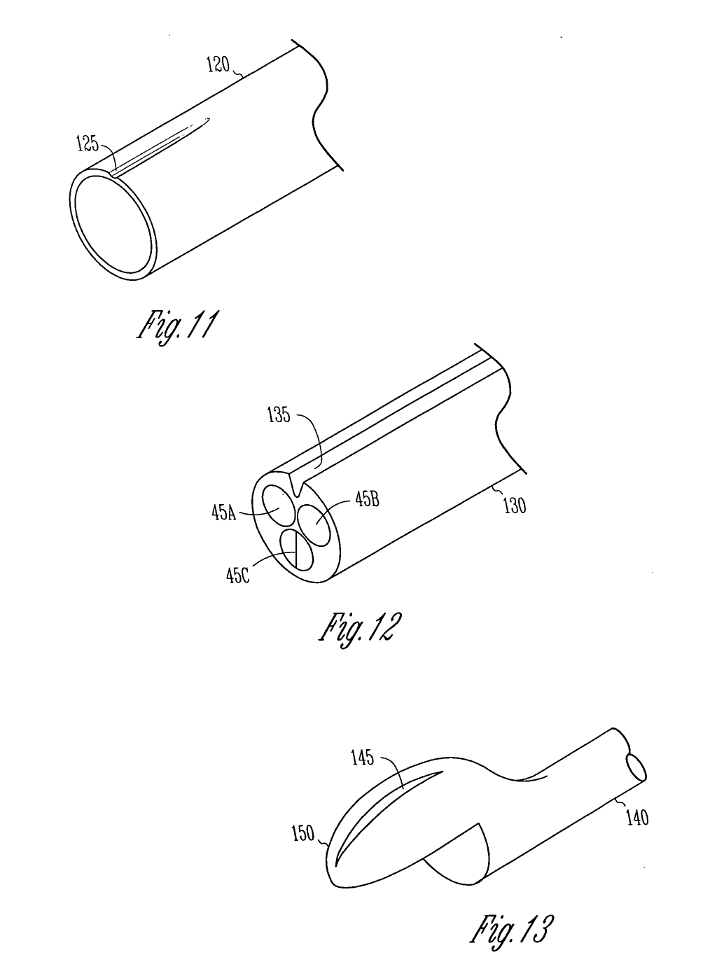

FIG. 8 illustrates shaft 100 having two bumps or raised dimples 105. Raised dimples 105 are formed by machining, molding, or bonding additional structure on shaft 100. Guide wire 70 can be engaged by the raised portions of dimples 105, as shown in the figure. FIG. 9 illustrates an embodiment wherein shaft 100 includes four raised dimples 105 however, greater or fewer numbers of raised dimples are also contemplated. For example, FIG. 10 illustrates an end view of single raised dimple 105 on shaft 110. In the figure, guide wire 70 is trapped by the combination of shaft 110, dimple 105 and an interior surface of ureter 15.

In various examples, raised dimples are generally conical or have a blade or ridge shape. The dimples are sufficiently smooth to reduce injury and allow easy passage in the ureter.

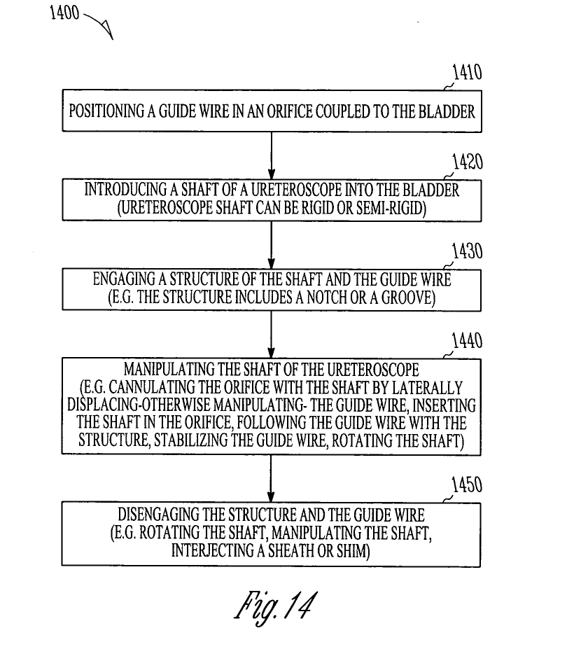

FIG. 11 illustrates shaft 120 having notch 125 cut in an outer surface. Notch 120 encroaches on the wall thickness of shaft 120 and as such, shaft 120 has a non-uniform wall thickness at the distal end.

FIG. 12 illustrates multi-lumen 45C having two channels wherein one channel provides, for example, irrigation and a second channel provides drainage. In addition, notch 135 is shown to extend along the length of shaft 130. Notch 135 can provide additional drainage and facilitate engagement of a guide wire.

FIG. 13 illustrates notch 145 on a surface of beak 150 coupled to shaft 140. Notch 145, in one example, includes a recessed groove to engage a guide wire.

The notch, magnet, dimples, blades or other structure of the present subject matter enhances the ability to traverse the bladder orifice as well as manipulate the guide wire during ureteroscopy procedures. The notch can be used to follow at any point along the length of the ureter such as to pass or circumvent an obstacle or other tortuous structure.

In addition, the present subject matter can be implemented in other instruments that are used with guide wires or other filamentous structures. For example, a linear tissue structure can be manipulated with a notch or dimple as described herein or used to guide manipulation of a tool or other instrument.

It is to be understood that the above description is intended to be illustrative, and not restrictive. For example, the above-described embodiments (and/or aspects thereof) may be used in combination with each other. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The Abstract of the Disclosure is provided to comply with 37 C.F.R. §1.72(b), requiring an abstract that will allow the reader to quickly ascertain the nature of the technical disclosure. It is submitted with the understanding that it will not be used to interpret or limit the scope or meaning of the claims. In addition, in the foregoing Detailed Description, various features may be grouped together to streamline the disclosure. This method of disclosure is not to be interpreted as reflecting an intention that the claimed embodiments require more features than are expressly recited in each claim. Rather, as the following claims reflect, inventive subject matter may lie in less than all features of a single disclosed embodiment. Thus the following claims are hereby incorporated into the Detailed Description, with each claim standing on its own as a separate embodiment.

1-5. (canceled)

6. A method comprising:
introducing a shaft of a ureteroscope into a bladder;
engaging a notch at an end of the shaft with a guide wire, the guide wire disposed in an orifice coupled to the bladder, and when engaged with the guide wire, the notch is operable to stabilize the guide wire;
using the notch to manipulate the guide wire to cannulate the orifice; and
using the notch to follow the guide wire.

7. The method of claim 6 further including disengaging the notch by rotating the shaft about a longitudinal axis of the shaft.

8-13. (canceled)

14. The method of claim 6 further including inserting the shaft in the orifice.

15. The method of claim 6 further including manipulating an end of the ureteroscope, the shaft of which is rigid or semi-rigid.

16. The method of claim 6 further including manipulating the guide wire using the ureteroscope.

17. A method comprising:
positioning a guide wire in an orifice of a bladder;
engaging a notch of a shaft of a ureteroscope with the guide wire;
using the notch to manipulate the guide wire to open the orifice; and
using the notch to follow the guide wire.

18. The method of claim 17 wherein engaging includes changing position or orientation of the shaft.

19. The method of claim 17 further including disengaging the notch from the guide wire.

20. The method of claim 19 wherein disengaging includes manipulating the shaft.

21. The method of claim 19 wherein disengaging includes rotating the shaft.

22. The method of claim 19 wherein disengaging includes interjecting at least one of a sheath and a shim between the guide wire and the shaft.

23. A method of introducing a ureteroscope in an orifice of a ureter, the orifice having a guide wire positioned therein, the method comprising:
engaging a structure of a shaft of the ureteroscope with the guide wire;
following the guide wire with the shaft using the structure;
using the structure to manipulate the guide wire to open the orifice;
inserting the shaft in the orifice; and
disengaging the structure and the guide wire.

24. The method of claim 23 wherein engaging the structure includes engaging at least one of a notch and a groove.

25. The method of claim 23 wherein engaging the structure includes stabilizing the guide wire.

26. The method of claim 23 wherein manipulating the guide wire includes using the structure to apply a lateral force on the guide wire.

27. The method of claim 23 wherein disengaging includes rotating the structure about the guide wire.
